(19) United States
(12) Patent Application Publication
(10) Pub. No.: US 2010/0048621 A1
(43) Pub. Date: Feb. 25, 2010

(54) MEDICAMENT FOR TREATING CHRONIC OBSTRUCTIVE PULMONARY DISEASE

(76) Inventors: Yoichi Yabuuchi, Tokushima-ken (JP); Kounori Kotosai, Tokushima-ken (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/530,422

(22) PCT Filed: Mar. 7, 2008
(86) PCT No.: PCT/JP2008/054689

§ 371 Date: Sep. 8, 2009

(30) Foreign Application Priority Data
Mar. 9, 2007 (JP) 2007-059664

(51) Int. Cl. A61K 31/4709; C07D 215/227; A61P 11/00

(52) U.S. Cl. 514/312; 546/153

(57) ABSTRACT

The invention relates to a medicament for treating chronic obstructive pulmonary disease comprising as active ingredients a carbostyril derivative of formula (1): wherein A is a lower alkylene group, R is a cycloalkyl group, the bonding between 3- and 4-positions of the carbostyril skeleton is a single bond or a double bond, or a salt thereof; and probucol.

### TECHNICAL FIELD

The invention relates to a medicament for treating chronic obstructive pulmonary disease, particularly a medicament for treating chronic obstructive pulmonary disease (COPD) comprising as active ingredients a carbostyril derivative of formula (1):

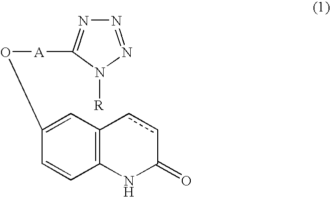

wherein A is a lower alkylene group, R is a cycloalkyl group, the bonding between 3- and 4-positions of the carbostyril skeleton is a single bond or a double bond, or a salt thereof; and probucol.

### BACKGROUND ART

The carbostyril derivatives of formula (1) or salts thereof and the process for the preparation thereof are disclosed in JP-63-20235-B and JP-55-35019-A. And it is known that the compounds have platelet aggregation inhibition action, phosphodiesterase (PDE) inhibition action, antiulcer, hypotensive action and antiphlogistic action, and are useful as an antithrombotic agent, a drug for improving cerebral circulation, an antiinflammatory agent, an antiulcer drug, an antihypertensive drug, an antiasthmatic drug, a phosphodiesterase inhibitor, etc. In addition, it is known that the compounds are also useful as a medicament for treating allergic disease (JP-5-320050-A). Furthermore, the compounds are also known as a medicament for treating COPD (JP-10-175864-A).

COPD (Chronic Obstructive Pulmonary Disease) is currently the fourth most common cause of death in America, which follows heart failure, cerebral infarction, and cancer. COPD tends to increase in the world in spite of the fact that the other diseases are decreasing, and additionally the number of the potential patients of COPD is large. Thus, it is foreseeable that the number will be increasing more and more from this time forward. The causes of COPD include abnormal inflammatory response due to smoking, adverse gas such as polluted air, or adverse microparticle, and thus COPD is a disease characterized by a progressive airflow limitation in connection with chronic obstructive bronchitis and emphysema.

Now, bronchodilators such as an anticholinergic agent and a β2 receptor agonist are clinically used as a medicament for treating COPD, however, the medicaments are not beyond palliative therapy and thus not basic medicaments for treating COPD. In addition, the anti-inflammatory agent usually used in acute exacerbation is a steroid drug, however, the utility thereof has not been satisfactorily studied yet.

The invasion in inflammatory cells of lung is one of main factors in chronic inflammation of COPD, and additionally oxidative stress caused by inflammatory cells, pulmonary cells impaired with inflammatory cells, or disorder material itself also relate to the chronic inflammation. Therefore, it is expectable that the control of the abnormal oxidative stress caused in inflammatory site will suppress the chronic inflammation and thus a new medicament preventing the pathologic progress will be developed. In addition, in a lung which is inflammatory site of COPD, it is known that elastase released from neutrophil which intrudes into pulmonary alveolus highly relates to the onset of emphysema, and it is also known that neutrophil elastase in blood is inactivated with α1-antitrypsin (α1-AT). Therefore, with such oxidative stress, α1-AT is oxidized to be inactivated, and thereby neutrophil elastase is not inactivated to give rise to tissue disorder. Thus, it is thought that a medicament for controlling such oxidative stress will prevent the pathologic progress.

As a pathologic model animal of COPD, an animal that suffers from emphysema by making the animal exposed to smoke of cigarettes for long period or by administering a variety of protease such as pig pancreas elastase (PPE) and human neutrophil elastase intratracheally, and an animal that suffers from emphysema via pulmonary disorder/inflammation by stimulating it with a variety of chemical materials such as LPS, cadmium chloride, nitrogen dioxide, ozone, and inorganic dust are disclosed. In addition, spontaneously malformed mice that is easy to suffer from emphysema such as Tight Skin (Tsk±) and Pallid (C57BL/6J pa+/pa+), or gene-manipulated mice such as Transgenic and Gene Targeting are also used.

### DISCLOSURE OF INVENTION

Thus, although some medicaments for treating COPD are clinically used as mentioned above, a more effective medicament for treating COPD is still desired since the current medicaments are not beyond palliative therapy.

The present inventors have intensively studied a new medicament for treating COPD, and have found that a combination or a drug combination of a carbostyril derivative of the above formula (1), especially 6-[4-(1-cyclohexyl-1H-tetrazol-5-yl)butoxy]3,4-dihydrocarbostyril (cilostazol) or a salt thereof, and probucol exhibits an excellent synergistic action for treating COPD. Especially, the combination also has some actions that can decrease the side effects of each medicament, depress an attack as the administration of steroid is required, improve the condition of COPD potently, and decrease the dose of a steroid for systemic administration. In addition, the combination or drug combination can be administered for long period thanks to its fast action and low toxicity. In addition, it is already known that cilostazol exhibits a bronchodilating action, and hence it is thought that this action can effectively act on improving the condition of COPD in the treatment with the combination or drug combination. The present invention is a useful medicament for treating COPD from the viewpoint of the safe treatment.

The present invention provides a medicament for treating chronic obstructive pulmonary disease comprising a carbostyril derivative of the general formula:

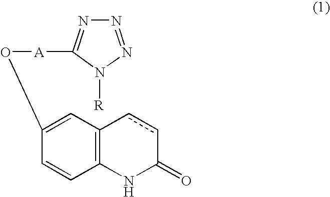

wherein A is a lower alkylene group, R is a cycloalkyl group, the bonding between 3- and 4-positions of the carbostyril skeleton is a single bond or a double bond, or a salt thereof, and probucol as active ingredients.

The present invention also provides a medicament for treating chronic obstructive pulmonary disease comprising 6-[4-(1-cyclohexyl-1H-tetrazol-5-yl)butoxy]3,4-dihydrocarbostyril (cilostazol) or a salt thereof, and probucol as active ingredients.

The present invention also provides a composition for treating chronic obstructive pulmonary disease comprising the above-mentioned ingredients.

The present invention also provides use of the carbostyril derivative or a salt thereof as mentioned above, and probucol in preparation of a medicament for treating chronic obstructive pulmonary disease.

The present invention also provides a method for treating chronic obstructive pulmonary disease which comprises administering an effective amount of the carbostyril derivative or a salt thereof as mentioned above, and probucol to a patient in need of such treatment.

According to the present invention, it is potently effective against COPD to use a carbostyril derivative (1), especially 6-[4-(1-cyclohexyl-1H-tetrazol-5-yl)butoxy]-3,4-dihydrocarbostyril, or a salt thereof, together with probucol.

### BRIEF DESCRIPTION OF DRAWINGS

FIG. 1 shows the combination effect affecting the gauge of pulmonary alveolus using COPD model mice treated with elastase.

### BEST MODE FOR CARRYING OUT THE INVENTION

The carbostyril derivative which is comprised as an ingredient of the drug combination or used in the combination use is a tetrazolylalkoxy-dihydrocarbostyril derivative of the formula:

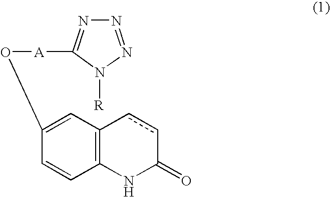

wherein

A is a lower alkylene group, R is a cycloalkyl group, the bonding between 3- and 4-positions of the carbostyril skeleton is a single bond or a double bond, or a salt thereof.

In the above formula (1), the cycloalkyl group includes C₃-C₈ cycloalkyl groups such as cyclopropyl, cyclobutyl, cyclopentyl, cyclohexyl, cycloheptyl, and cyclooctyl. Preferred cycloalkyl group is cyclohexyl. The lower alkylene group includes C₁-C₆ alkylene groups such as methylene, ethylene, propylene, tetramethylene, butylene, and pentylene, among which preferred one is tetramethylene.

Preferable carbostyril derivative is 6-[4-(1-cyclohexyl-1H-tetrazol-5-yl)butoxy]-3,4-dihydrocarbostyril, which has been put on the market in the trade name of cilostazol as an antiplatelet agent.

The carbostyril derivative (1) can be easily converted to a salt thereof by getting it treated with a pharmaceutically acceptable acid. The acid includes, for example, inorganic acids such as hydrochloric acid, sulfuric acid, phosphoric acid, and hydrobromic acid; and organic acids such as oxalic acid, maleic acid, fumaric acid, malic acid, tartaric acid, citric acid, and benzoic acid.

These carbostyril derivatives (1) and salts thereof and processes for preparation thereof are disclosed in JP-55-35019-A (relevant to U.S. Pat. No. 4,277,479).

The other active ingredient, Probucol is a compound having a chemical name of 4,4′-isopropylidenedithiobis[2,6-di-tert-butylphenol], and has already been put on the market as an anti-hyperlipidemic agent. It is also known that this compound has an activity inhibiting the production of oxidized LDL (low-density lipoprotein) (cf. J. Clin. Invest., 77, p. 641, 1986)

These active ingredients, a carbostyril derivative (1) and probucol may be administered together or separately, at the same time or different time. These ingredients may usually be used in a conventional pharmaceutical formulation. Then, these ingredients may be prepared in a single dosage form or in separate dosage forms.

The dose of these active ingredients is not limited to a specific range. The carbostyril derivatives (1) or a salt thereof may be used in an amount of 50 to 200 mg/day per an adult (50 kg of body weight), which is administered once a day or two to several times per day. Probucol may be used in an amount of 100 to 1000 mg/day per an adult (50 kg of body weight), which may be administered once a day, but may preferably be administered two to several times per day. When these ingredients are prepared in a single dosage form, they are incorporated in a ratio of 0.25 to 10 parts by weight of probucol per 1 part by weight of the carbostyril derivative (1) or a salt thereof. And, the drug combination may include the sum of the ingredients in 0.1-70% (w/w) per the preparation, but not limited thereto.

The each dosage form used for the drug combination or the combination in the present invention includes, for example, the dosage forms exemplified in JP-10-175864-A, and typically an oral solid dosage form such as tablets and capsules, an oral liquid dosage form such as syrups and elixirs, a parenteral dosage form such as injections, and an inhalant.

The preparations of the invention such as tablets, capsules, liquid for oral administration may be prepared by a conventional method. The tablets may be prepared by mixing the active ingredient(s) with conventional pharmaceutical carriers such as gelatin, starches, lactose, magnesium stearate, talc, gum arabic, and the like. The capsules may be prepared by mixing the active ingredient(s) with inert pharmaceutical fillers or diluents and filling hard gelatin capsules or soft capsules with the mixture. The oral liquid preparations such as syrups or elixirs are prepared by mixing the active ingredient(s) with sweetening agents (e.g. sucrose), preservatives (e.g. methylparaben, propylparaben), colorants, flavors, and the like. The preparations for parenteral administration may also be prepared by a conventional method, for example, by dissolving the active ingredient(s) of the present invention in a sterilized aqueous carrier, preferably water or a saline solution. Preferred liquid preparation suitable for parenteral administration is prepared by dissolving the daily dose of the active ingredients as mentioned above in water and an organic solvent and further in a polyethylene glycol having a molecular weight of 300 to 5000, in which preferably a lubricant such as sodium carboxymethylcellulose, methylcellulose, polyvinyl-pyrrolidone, and polyvinyl alcohol is incorporated. Preferably, the above liquid preparations may further comprise a disinfectant (e.g. benzyl alcohol, phenol, thimerosal), a fungicide, and further optionally an isotonic agent (e.g. sucrose, sodium chloride), a topical anesthetic, a stabilizer, a buffer, and the like. In view of keeping stability, the preparation for parenteral administration may be put in capsules, followed by removing the aqueous medium by a conventional lyophilizing technique. The preparation can be recovered into a liquid preparation by dissolving in an aqueous medium when used. The inhalants may be prepared by a conventional method. That is, the inhalants may be prepared by getting an active compound to a powder or liquid state, mixing it into propellants and/or carriers for inhalant, and charging an appropriate vaporizer with the mixture. Ordinarily, a mechanical powder vaporizer can be used when the active compound is a powder, and a vaporizer such as a nebulizer can be used when the compound is a liquid. In addition, the inhalant may optionally comprise a surfactant, an oil, a flavor, a cyclodextrin or a derivative thereof which has been used when necessary.

The examples of the above-mentioned additive agents include, but not limited thereto, what JP-10-175864-A discloses.

### Example

Combination effect of cilostazol/probucol for pulmonary disorder in C57BL/6J mouse treated with elastase produced from human neutrophil.

### Method of Experiment

Animals: female C57BL/6J mice (5 weeks old) which were purchased from Charles River Laboratories Japan, Inc. were used.

Group composition: the following 5 groups.
Non-treated (normal control) group: n=4
Elastase-treated group (control group): n=6
Elastase-treated, 0.3% cilostazol-administrated group: n=6
Elastase-treated, 0.5% probucol-administrated group: n=6
Elastase-treated, (0.3% cilostazol+0.5% probucol)−administrated group: n=6

Female C57BL/6J mice (5 weeks old) were divided into the given groups by a stratified randomization (using an SAS software, R 8.1) based on each body weight on the starting day of administration. Shortly after the group division, a MF feed was given to the non-treated group and the control group, and a mixed feed in which cilostazol or/and probucol was added in the given ratio was given to the medicament(s)-treated groups, in free feeding. On 7th day after the administration started, elastase produced from human neutrophil (Elastin Products Co. Inc.) was intratracheally administered in a dose of 20 U/50 μL to pentobarbital-anesthetized mice from their larynges using a sprayer (Penn-Century Inc.). Three weeks after the elastase administration, the animals were sacrificed by bleeding from abdominal venae-cavae under ether anesthesia, and then lungs thereof were extirpated, which were perfusion-fixed with 10% neutral formalin buffer solution. The pulmonary tissue fixed with formalin was paraffin-embedded, sliced, and stained with Masson Trichrome staining and HE staining in the Biopathology institute Co., Ltd. The evaluation of the pathological tissue was carried out based on the mean linear intercept of pulmonary alveolus thereof which is an objective indicator of pulmonary alveolus disorder (M. S. Dunnill, Torax (1962), 17, 320).

### Statistical Analysis

The following groups were statistically analyzed in order to study about the effect of the single-administration and combination-administration of the medicament(s).
1) Elastase-treated group (control group)
2) 0.3% Cilostazol-administrated group
3) 0.5% Probucol-administrated group
4) (0.3% Cilostazol+0.5% probucol)−administrated group

In order to evaluate the combination effect, Two-Way Analysis of Variance was carried out between the elastase-treated group, and the cilostazol-administrated group, the probucol-administrated group or the combination administrated group to test each interaction.

Dunnett tests of the cilostazol-administrated group and the probucol-administrated group against the elastase-treated group were carried out. In addition, Dunnett tests of the cilostazol-administrated group and the probucol-administrated group against the combination-administrated group were also carried out.

All tests were done by two-sided test with significant level of 5%. The test was carried out using a SAS software (SAS Institute Japan, R 8.1).

### Results

Combination effect affecting mean linear intercept of pulmonary alveolus in elastase-treated model mouse of COPD.

All the results of 0.3% cilostazol-administrated group (116.9±14.3 μm), 0.5% probucol-administrated group (86.2±4.8 μm), and (0.3% cilostazol+0.5% probucol)−administrated group (67.5±3.7 μm) exhibited significant inhibitory-effects against that of the elastase-treated group (control group, 178.8±22.4 μm) (mean±standard deviation, P<0.01), and furthermore the result of the combination-administrated group exhibited significant meliorating-effect to reach the same level as the non-treated (normal control) group (51.4±1.9 μm).

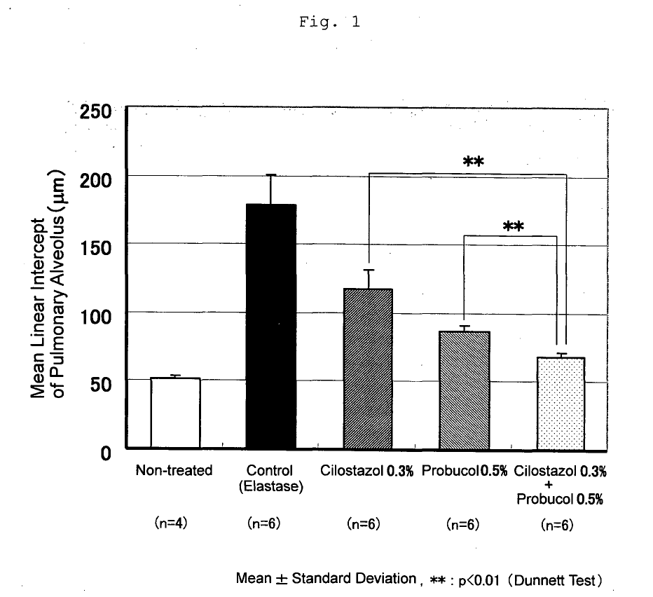

In the comparison between each single-administration and the combination-administration, the combination-administration exhibited more significant decreasing-effect of the mean linear intercept of pulmonary alveolus than the single-administration of either 0.5% probucol-administrated group or 0.3% cilostazol-administrated group did (FIG. 1).

1. A medicament for treating chronic obstructive pulmonary disease comprising a carbostyril derivative of the formula: 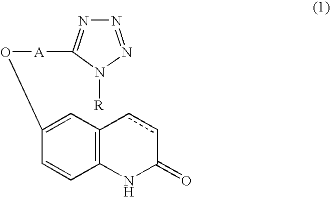
wherein
A is a lower alkylene group, R is a cycloalkyl group, the bonding between 3- and 4-positions of the carbostyril skeleton is a single bond or a double bond, or a salt thereof, and probucol as active ingredients.

2. The medicament of claim 1 wherein the carbostyril derivative is 6-[4-(1-cyclohexyl-1H-tetrazol-5-yl)butoxy]-3,4-dihydrocarbostyril or a salt thereof.

3. Use of the carbostyril derivative or a salt thereof as set forth in claim 1 or 2, and probucol in preparation of a medicament for treating chronic obstructive pulmonary disease.

4. A method for treating chronic obstructive pulmonary disease which comprises administering an effective amount of the carbostyril derivative or a salt thereof as set forth in claim 1 or 2, and probucol to a patient in need of such treatment.
